(19) United States
(12) Patent Application Publication
(10) Pub. No.: US 2006/0116420 A1
(43) Pub. Date: Jun. 1, 2006

(54) Crystalline forms of 3'-tert-Butyl-3'-N-tert-butyloxycarbonyl-4-deacetyl-3'-dephenyl-3'-N-debenzoyl-4-O-methoxycarbonyl-paclitaxel

(76) Inventors: Ramakrishnan Chidambaram, Pennington, NJ (US); Robert Kevin Perrone, Belle Mead, NJ (US); Martha Davidovich, East Brunswick, NJ (US); John D. DiMarco, East Brunswick, NJ (US); Gary McGeorge, Franklin Park, NJ (US); Imre M. Vitez, Whitehouse Station, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/285,463

(22) Filed: Nov. 22, 2005

Related U.S. Application Data
(60) Provisional application No. 60/630,225, filed on Nov. 23, 2004.

(51) Int. Cl. A61K 31/337; C07D 405/14

(52) U.S. Cl. 514/449; 549/510

(57) ABSTRACT

The present invention relates to crystalline forms of 3′-tert-Butyl-3′-N-tert-butyloxycarbonyl-4-deacetyl-3 ′-dephenyl-3′-N-debenzoyl-4-O-methoxycarbonyl-paclitaxel represented by formula I; 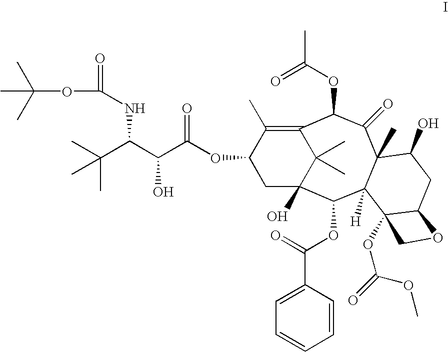 processes for the production thereof; pharmaceutical compositions thereof; methods for preparing the pharmaceutical composition; and methods for inhibiting tumor growth therewith.

### RELATED APPLICATIONS

This application claims the benefit of U.S. Provisional Application No. 60/630,225, filed Nov. 23, 2004, incorporated herein by reference in its entirety.

### FIELD OF THE INVENTION

The present invention relates to crystalline forms of 3′-tert-Butyl-3′-N-tert-butyloxycarbonyl-4-deacetyl-3′-dephenyl-3′-N-debenzoyl-4-O-methoxycarbonyl-paclitaxel; processes for the production thereof; pharmaceutical compositions thereof; methods for preparing the pharmaceutical composition; and methods for inhibiting tumor growth therewith.

### BACKGROUND OF THE INVENTION

Paclitaxel is a natural product extracted from the bark of Pacific yew trees, Taxus brevifolia, and is the active constituent of the anticancer agent TAXOL®. It has been shown to have excellent antitumor activity in in vivo animal models, and recent studies have elucidated its unique mode of action, which involves abnormal polymerization of tubulin and disruption of mitosis. It is used clinically against a number of human cancers. It is an important cancer agent both therapeutically and commercially. Numerous clinical trials are in progress to expand and increase the utility of this agent for the treatment of human proliferative diseases. The results of TAXOL® clinical studies have been reviewed by numerous authors. A very recent compilation of articles by a number of different authors is contained in the entire issue of Seminars in Oncology 1999, 26 (1, Suppl 2). Other examples include articles by Rowinsky et al. in TAXOL®: A Novel Investigational Antimicrotubule Agent, J. Natl. Cancer Inst., 82: pp 1247-1259, 1990; by Rowinsky and Donehower in “The Clinical Pharmacology and Use of Antimicrotubule Agents in Cancer Chemotherapeutics,” Pharmac. Ther., 52:35-84, 1991; by Spencer and Faulds in “Paclitaxel, A Review of its Pharmacodynamic and Pharmacokinetic Properties and Therapeutic Potential in the Treatment of Cancer,” Drugs, 48 (5) 794-847, 1994; by K. C. Nicolaou et al. in “Chemistry and Biology of TAXOL®,” Angew. Chem., Int. Ed. Engl., 33: 15-44, 1994; by F. A. Holmes, A. P. Kudelka, J. J. Kavanaugh, M. H. Huber, J. A. Ajani, V. Valero in the book “Taxane Anticancer Agents Basic Science and Current Status” edited by Gunda I. Georg, Thomas T. Chen, Iwao Ojima, and Dolotrai M. Vyas, 1995, American Chemical Society, Washington, D.C., 31-57; by Susan G. Arbuck and Barbara Blaylock in the book “TAXOL® Science and Applications” edited by Mathew Suffness, 1995, CRC Press Inc., Boca Raton, Fla., 379-416; and also in the references cited therein.

A semi-synthetic analog of paclitaxel named docetaxel has also been found to have antitumor activity and is the active ingredient of the commercially available cancer agent TAXOTERE®. See, Biologically Active Taxol Analogues with Deleted A-Ring Side Chain Substitutents and Variable C-2′ Configurations, J. Med. Chem., 34, pp 1176-1184 (1991); Relationships between the Structure of Taxol Analogues and Their Antimitotic Activity, J. Med. Chem., 34, pp 992-998 (1991). A review of the clinical activity of TAXOTERE® by Jorge E. Cortes and Richard Pazdur has appeared in Journal of Clinical Oncology 1995, 13(10), 2643 to 2655. The structures of paclitaxel and docetaxel are shown below along with the conventional numbering system for molecules belonging to the class; such numbering system is also employed in this application. 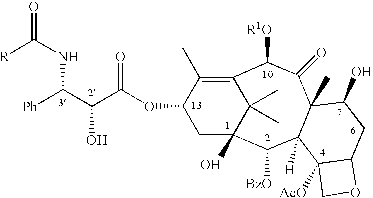
paclitaxel (TAXOL®): R=Ph; R′=acetyl
docetaxel (TAXOTERE®): R=t-butoxy; R′=hydrogen

U.S. Pat. No. 6,750,246 describes C-4 methyl carbonate taxane analogs which have been shown to possess surprising oral activity and thus would have utility against proliferative diseases after oral administration. WO 03/053350 discloses pharmaceutical compositions of orally effective taxane derivatives and to their use for inhibiting tumor growth in mammalian hosts. The entire disclosures of each of the aforementioned patents and patent publications are incorporated herein by reference.

A particularly advantageous C-4 methyl carbonate taxane analog that has been found to have superior oral activity is 3′-tert-Butyl-3′-N-tert-butyloxycarbonyl-4-deacetyl-3′-dephenyl-3′-N-debenzoyl-4-O-methoxycarbonyl-paclitaxel, having the structure of formula I:

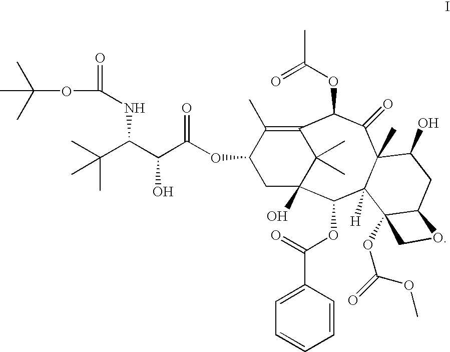

In accordance with the present invention, the 3′-tert-Butyl-3′-N-tert-butyloxycarbonyl-4-deacetyl-3′-dephenyl-3′-N-debenzoyl-4-O-methoxycarbonyl-paclitaxel is provided in crystalline form, including polymorphs which have been designated as Form N-2 and Form THF-1 described further hereinbelow.

### BRIEF DESCRIPTION OF THE DRAWINGS

FIG. 1 is a powder x-ray diffraction pattern for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 2 is a powder x-ray diffraction pattern for Form THF-1 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 3 is a DSC thermogram for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 4 is a DSC thermogram for Form THF-1 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 5 is a TGA thermogram for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 6 is a TGA thermogram for Form THF-1 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 7 is a carbon-13 CP-MAS SSNMR spectrum for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 8 is a Raman spectrum for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

FIG. 9 is an IR spectrum for Form N-2 of the 3′-tert-Butyl-3′-N-tert-butyloxycarbonyl-4-deacetyl-3′-dephenyl-3′-N-debenzoyl-4-O-methoxycarbonyl-paclitaxel.

### SUMMARY OF THE INVENTION

In accordance with the present invention, there is provided the 3′-tert-Butyl-3′-N-tert-butyloxycarbonyl-4-deacetyl-3′-dephenyl-3′-N-debenzoyl-4-O-methoxycarbonyl-paclitaxel represented by the following formula I in crystalline form:

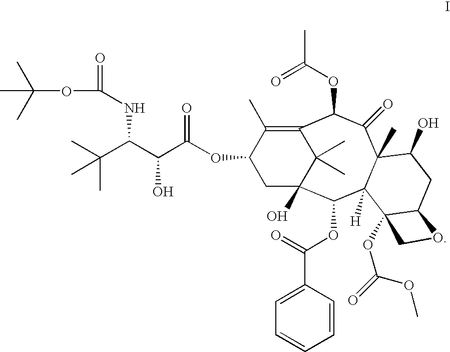

The present invention is further directed to crystalline polymorphs of the formula I taxane, designated as Form N-2 and Form THF-1, as well as mixtures thereof. The present invention further pertains to processes for the production of the polymorphs; pharmaceutical compositions thereof; methods for preparing the pharmaceutical composition; and the use of these crystalline forms in the treatment of cancers and other proliferating diseases.

### DETAILED DESCRIPTION OF THE INVENTION

The present invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, depicted hereinbelow as the compound formula I. The invention also provides a crystalline form of 3′-tert-Butyl-3′-N-tert-butyloxycarbonyl-4-deacetyl-3′-dephenyl-3′-N-debenzoyl-4-O-methoxycarbonyl-paclitaxel which is substantially pure, i.e., its purity greater than about 90%.

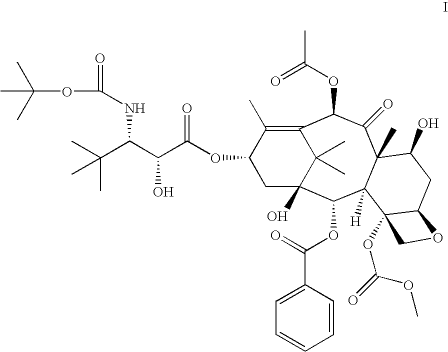

The crystalline forms of the instant invention can be characterized using X-Ray Powder Diffraction (XRPD), Differential Scanning Calorimetry (DSC), Thermogravimetric Analysis (TGA), carbon-13 Solid State Nuclear Magnetic Resonance (SSNMR), Raman Spectroscopy and Infrared Spectroscopy (IR). It is to be understood that numerical values described and claimed herein are approximate. Variation within the values may be attributed to equipment calibration, equipment errors, purity of the materials, crystals size, and sample size, among other factors. In addition, variation may be possible while still obtaining the same result. For example, X-ray diffraction values are generally accurate to within ±0.2 degrees and intensities (including relative intensities) in an X-ray diffraction pattern may fluctuate depending upon measurement conditions employed. Similarly, DSC results are typically accurate to within about 2° C. Also, carbon-13 SSNMR results are generally accurate to within about ±0.2 ppm. Consequently, it is to be understood that the crystalline forms of the instant invention are not limited to the crystalline forms that provide characterization patterns (i.e., one or more of the XRPD, DSC, TGA, SSNMR, Raman and IR) completely identical to the characterization patterns depicted in the accompanying Figures disclosed herein. Any crystalline forms that provide characterization patterns substantially the same as those described in the accompanying Figures fall within the scope of the present invention. The ability to ascertain substantially the same characterization patterns is within the purview of one of ordinary skill in the art.

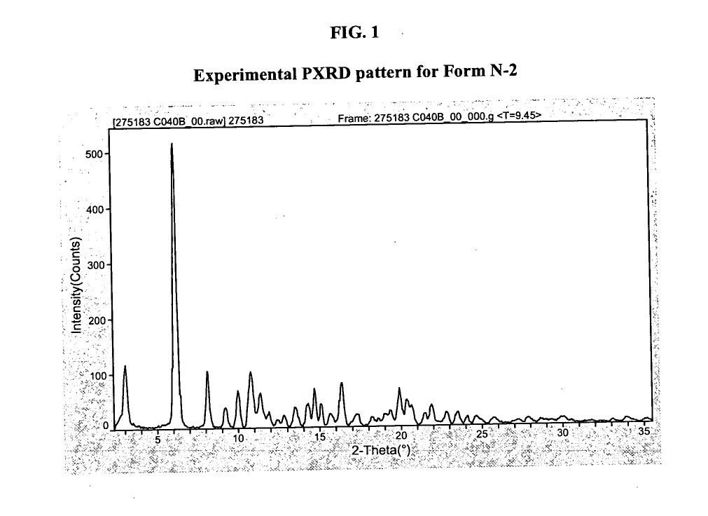

In one aspect of the invention, there is provided a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits an XRPD pattern substantially the same as that depicted in FIG. 1, comprising one or more 20 values selected from: 3.0±0.2, 6.2±0.2, 8.1±0.2, 9.2±0.2, 10.0±0.2, 13.5±0.2 and 16.4±0.2. The invention also provides a Form N-2 crystal that exhibits an XRPD pattern having characteristic diffraction peaks expressed in degrees 2-theta, diffraction d-spacings expressed in Å, and intensities (I), at approximately the values shown in Table 1 hereinbelow.

TABLE 1
3′-tert-Butyl-3′-N-tert-butyloxycarbonyl-4-deacetyl-3′-dephenyl-
3′-N-debenzoyl-4-O-methoxycarbonyl-paclitaxel, Form N-2

| 2-Theta | d(A) | I % |
|---|---|---|
| 3.045 | 28.9894 | 21.6 |
| 6.194 | 14.2568 | 100.0 |
| 8.112 | 10.8896 | 19.4 |
| 9.203 | 9.6010 | 6.2 |
| 10.002 | 8.8362 | 11.8 |
| 10.802 | 8.1834 | 17.9 |
| 11.390 | 7.7621 | 10.7 |
| 11.889 | 7.4376 | 4.1 |
| 12.803 | 6.9085 | 3.1 |
| 13.465 | 6.5704 | 5.8 |
| 14.294 | 6.1911 | 7.0 |
| 14.699 | 6.0216 | 12.0 |
| 15.103 | 5.8613 | 6.0 |
| 16.386 | 5.4051 | 14.0 |
| 17.301 | 5.1214 | 3.3 |
| 19.904 | 4.4571 | 10.3 |
| 20.392 | 4.3514 | 6.8 |
| 21.465 | 4.1363 | 3.3 |
| 21.896 | 4.0559 | 6.2 |
| 22.815 | 3.8945 | 3.3 |
| 23.532 | 3.7775 | 3.3 |

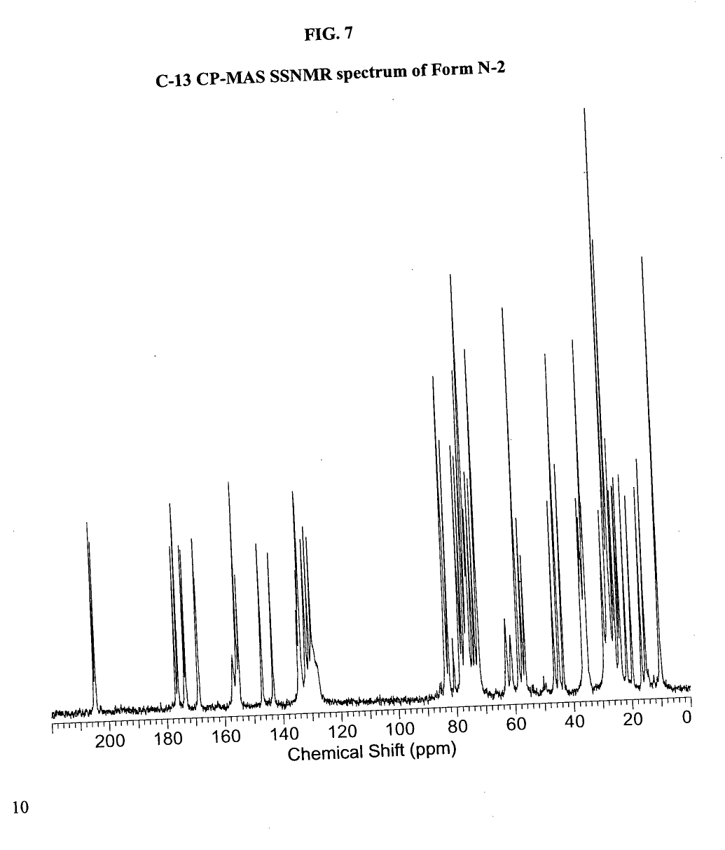

In another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits SSNMR carbon-13 chemical shifts substantially the same as shown in FIG. 7. The invention also provides a Form N-2 crystal that exhibits SSNMR carbon-13 chemical shifts as a powder expressed in part per million at approximately 10.1, 15.1, 16.4, 19.7, 21.5, 23.5, 24.1, 25.2, 25.7, 26.8, 27.6, 29.1, 35.0, 35.3, 36.0, 36.3, 36.6, 43.4, 45.0, 46.5, 56.4, 57.3, 58.7, 61.0, 62.6, 80.7, 82.5, 83.3, 83.6, 129.1, 129.7, 130.6, 131.7, 133.4, 133.5, 133.9, 134.4, 143.2, 147.0, 154.8, 155.5, 156.9, 168.7, 169.0, 173.1, 173.6, 175.9, and 176.7.

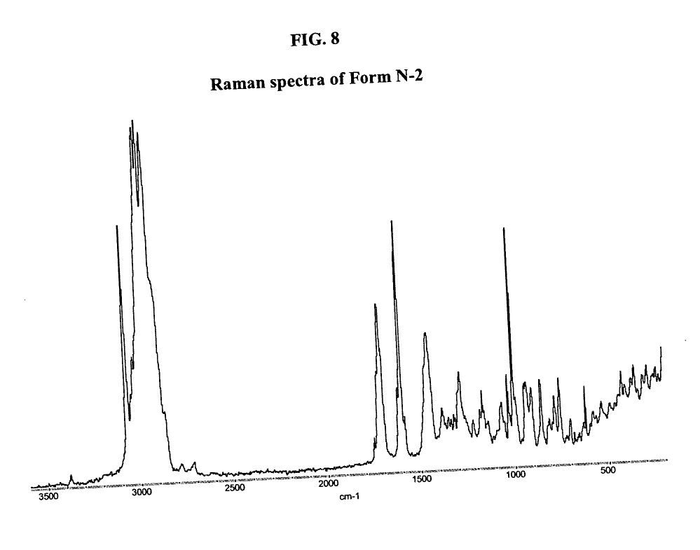

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits a Raman spectrum substantially the same as shown in FIG. 8. The invention also provides a Form N-2 crystal that exhibits a Raman spectrum comprising frequencies expressed in cm⁻¹ at approximately 3069, 3027, 2975, 2961, 2938, 2864, 1750, 1709, 1602, 1585, 1453, 1165, 1060, 1000, 904, 854, and 617.

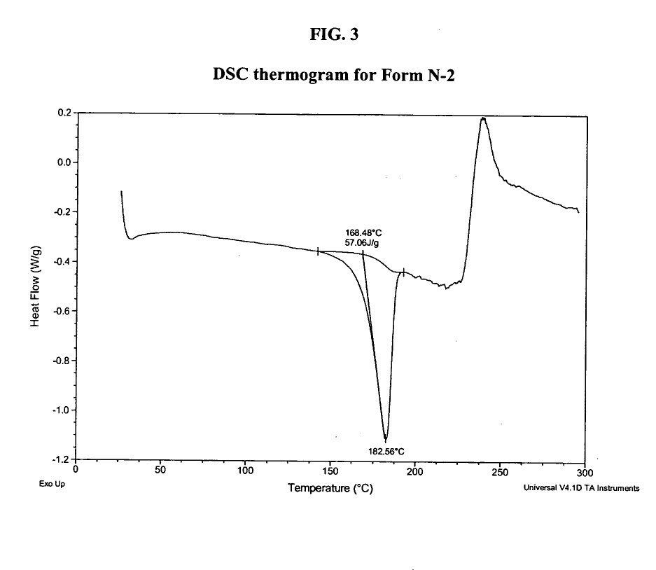

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits a differential scanning calorimetry (DSC) thermogram having a peak at about 168° C. to about 183° C. The invention also provides a Form N-2 crystal that exhibits a DSC thermogram substantially the same as shown in FIG. 3.

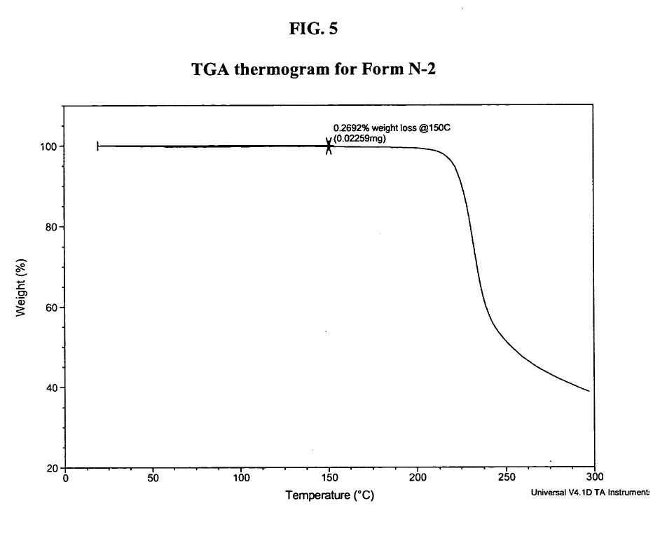

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits a thermogravimetric analysis (TGA) thermogram having minimal weight loss in accordance to a neat form, wherein about 0.3% weight loss was observed. The invention also provides a Form N-2 crystal that exhibits a TGA thermogram substantially the same as shown in FIG. 5.

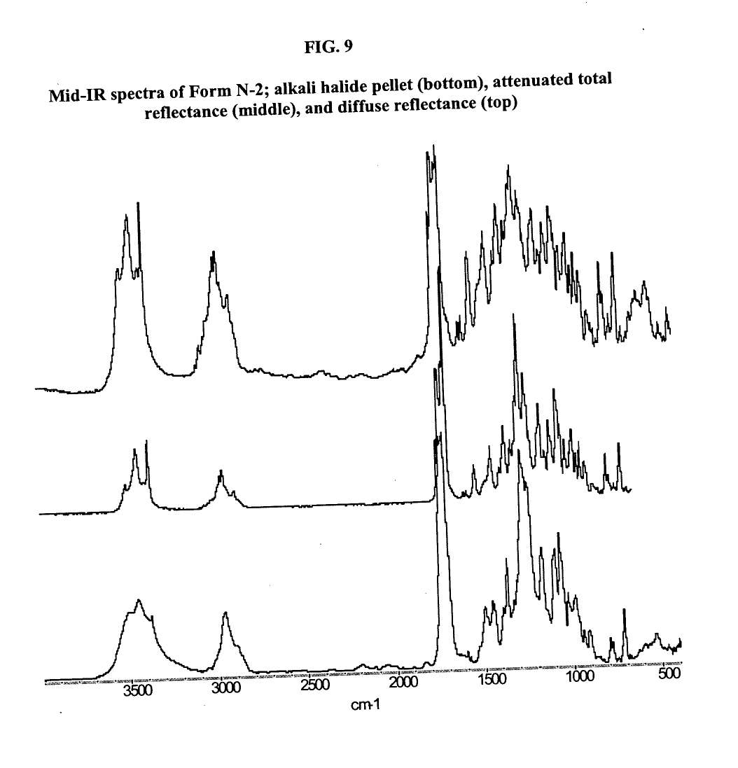

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form N-2, which exhibits IR spectra substantially the same as shown in FIG. 9.

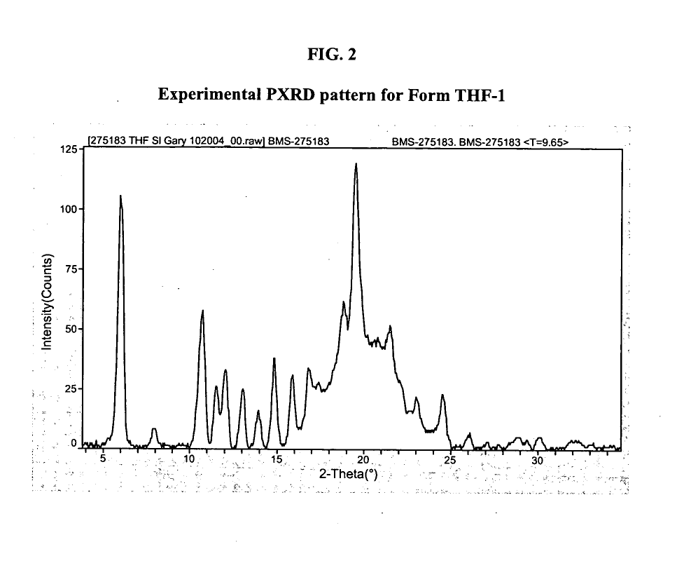

In a further aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form THF-1, which exhibits an XRPD pattern substantially the same as that depicted in FIG. 2, comprising one or more 20 values selected from: 6.0±0.2, 10.7±0.2, 11.5±0.2, 12.0±0.2, 16.8±0.2, and 19.5±0.2. The invention also provides a Form THF-1 crystal that exhibits an XRPD pattern having characteristic diffraction peaks expressed in degrees 2-theta, diffraction d-spacings expressed in Å, and intensities (I), at approximately the values shown in Table 2 hereinbelow.

TABLE 2
3′-tert-Butyl-3′-N-tert-butyloxycarbonyl-4-deacetyl-3′-dephenyl-
3′-N-debenzoyl-4-O-methoxycarbonyl-paclitaxel, Form THF-1

| 2-Theta | d(A) | I % |
|---|---|---|
| 5.958 | 14.8210 | 100.0 |
| 7.947 | 11.1159 | 6.7 |
| 10.743 | 8.2283 | 51.4 |
| 11.507 | 7.6838 | 21.0 |
| 12.049 | 7.3391 | 26.7 |
| 13.051 | 6.7779 | 21.9 |
| 13.949 | 6.3435 | 12.4 |
| 14.809 | 5.9772 | 34.3 |
| 15.889 | 5.5730 | 23.8 |
| 16.809 | 5.2701 | 23.8 |
| 18.802 | 4.7157 | 35.2 |
| 19.497 | 4.5491 | 74.3 |
| 21.499 | 4.1298 | 34.3 |
| 24.509 | 3.6290 | 18.1 |

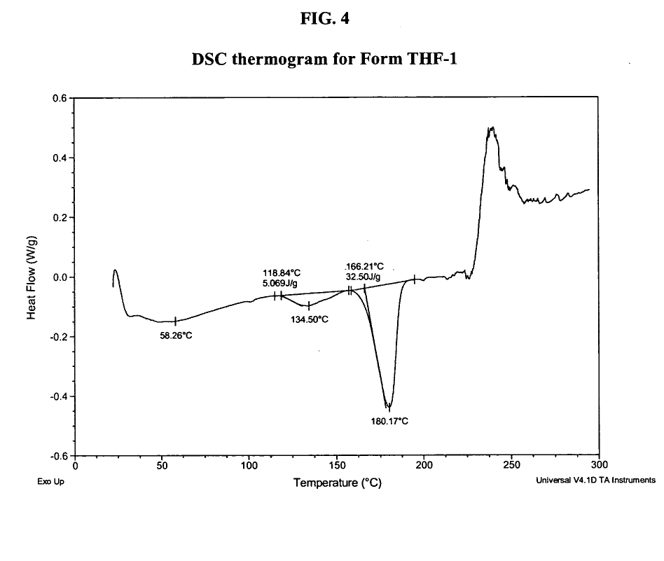

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form THF-1, which exhibits a differential scanning calorimetry (DSC) thermogram having a peak at about 166° C. to about 180° C. The invention also provides a Form THF-1 crystal that exhibits a DSC thermogram substantially the same as shown in FIG. 4.

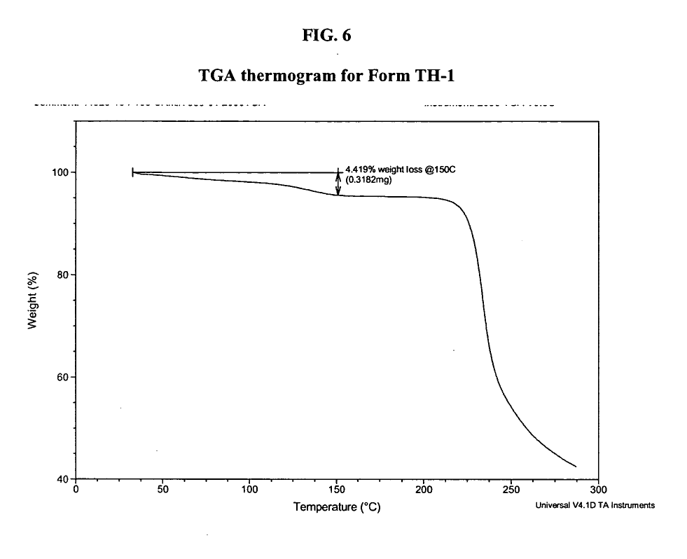

In yet another aspect, the invention provides a crystalline form of 3′-tert-Butyl-3′-N-tert-butyloxycarbonyl-4-deacetyl-3′-dephenyl-3′-N-debenzoyl-4-O-methoxycarbonyl-paclitaxel, designated as Form THF-1, which exhibits a thermogravimetric analysis (TGA) thermogram having an about 4.5% weight loss in accordance to a mono THF solvate form. The invention also provides a Form THF-1 crystal that exhibits a TGA thermogram substantially the same as shown in FIG. 6.

In a further aspect, the invention provides a process for preparing the aforementioned Form THF-1 crystals, which process comprises mixing the 3′-tert-Butyl-3′-N-tert-butyloxycarbonyl-4-deacetyl-3′-dephenyl-3′-N-debenzoyl-4-O-methoxycarbonyl-paclitaxel in a suitable aprotic solvent system with heating until dissolution is essentially complete to form a solution, followed by cooling the resulting solution to a lower temperature, preferably room temperature, to allow Form THF-1 crystals to crystallize. Preferred aprotic solvent systems comprise THF and an aprotic substantially THF-miscible co-solvent such as heptane, hexane, cyclohexane, toluene, or mixtures thereof. The use of THF and heptane is particularly preferred.

In yet another aspect, the invention provides a process for preparing the aforementioned Form N-2 crystals, which process comprises mixing the 3′-tert-Butyl-3′-N-tert-butyloxycarbonyl-4-deacetyl-3′-dephenyl-3′-N-debenzoyl-4-O-methoxycarbonyl-paclitaxel or Form THF-1 of the 3′-tert-Butyl-3′-N-tert-butyloxycarbonyl-4-deacetyl-3′-dephenyl-3′-N-debenzoyl-4-O-methoxycarbonyl-paclitaxel in a suitable aprotic solvent system with heating until dissolution is essentially complete to form a solution, followed by cooling the resulting solution to a lower temperature, preferably room temperature, to allow Form N-2 crystals to crystallize. Preferred aprotic solvent systems comprise (i) at least one solvent selected from ethyl acetate, isopropyl acetate, and toluene; and (ii) at least one solvent selected from heptane, hexane, and cyclohexane. The use of ethyl acetate and heptane is particularly preferred.

In a further aspect, the invention provides a process for preparing a pharmaceutical composition comprising the 3′-tert-Butyl-3′-N-tert-butyloxycarbonyl-4-deacetyl-3′-dephenyl-3′-N-debenzoyl-4-O-methoxycarbonyl-paclitaxel and at least one pharmaceutically acceptable carrier or excipient, which process comprises mixing Form N-2 and/or THF-1 crystals of 3′-tert-Butyl-3′-N-tert-butyloxycarbonyl-4-deacetyl-3′-dephenyl-3′-N-debenzoyl-4-O-methoxycarbonyl-paclitaxel with at least one said pharmaceutically acceptable carrier or excipient. Preferred processes comprise mixing the aforementioned Form N-2 crystals with at least one pharmaceutically acceptable carrier or excipient. Pharmaceutically acceptable carriers or excipients include, without limitation, polyether glycols, saturated or unsaturated polyglycolized glyceridea, solid amphiphilic surfactants, surfactants other than said solid amphiphilic surfactants, alcohols other than a polyether glycols, fatty acid ester derivatives of polyhydric alcohols, vegetable oils, mineral oils, and optionally, an effective amount of a pharmaceutically acceptable acid for enhancing the stability of the drug. It should be understood that the crystalline forms of Form N-2 and Form THF-1 may, in some cases, change to other form or forms (e.g., amorphous), or solublize, upon mixing with at least one pharmaceutically acceptable carrier or excipient.

In a yet another aspect, the invention provides a pharmaceutical composition comprising Form N-2 and/or Form THF-1 crystals of 3′-tert-Butyl-3′-N-tert-butyloxycarbonyl-4-deacetyl-3′-dephenyl-3′-N-debenzoyl-4-O-methoxycarbonyl-paclitaxel and at least one pharmaceutically acceptable carrier or excipient. A preferred pharmaceutical composition comprises the aforementioned Form N-2 crystals and at least one pharmaceutically acceptable carrier or excipient.

In a yet another aspect, the invention provides methods for inhibiting tumor growth which methods comprise administering to a mammal in need of such treatment crystals of Form N-2, Form THF-1, or mixtures thereof; or a pharmaceutical composition comprising crystals of Form N-2, Form THF-1, or mixtures thereof. A preferred crystal form useful in the practice of the instant methods of inhibiting tumor growth comprises 3′-tert-Butyl-3′-N-tert-butyloxycarbonyl-4-deacetyl-3′-dephenyl-3′-N-debenzoyl-4-O-methoxycarbonyl-paclitaxel Form N-2 and the preferred method of administering to a mammal using such Form N-2 crystals is oral.

The 3′-tert-Butyl-3′-N-tert-butyloxycarbonyl-4-deacetyl-3′-dephenyl-3′-N-debenzoyl-4-O-methoxycarbonyl-paclitaxel display a significant inhibitory effect with regard to abnormal cell proliferation, and have therapeutic properties that make it possible to treat patients who have pathological conditions associated with an abnormal cell proliferation. In addition, these compounds possess significant oral bioavailability and thus can elicit their positive therapeutic effects after being administered orally.

The pathological conditions include the abnormal cellular proliferation of malignant or non-malignant cells in various tissues and/or organs, including, non-limitatively, muscle, bone and/or conjunctive tissues; the skin, brain, lungs and sexual organs; the lymphatic and/or renal system; mammary cells and/or blood cells; the liver, digestive system, and pancreas; and the thyroid and/or adrenal glands. These pathological conditions can also include psoriasis; solid tumors; ovarian, breast, brain, prostate, colon, stomach, kidney, and/or testicular cancer, Karposi's sarcoma; cholangiocarcinoma; choriocarcinoma; neuroblastoma; Wilm's tumor, Hodgkin's disease; melanomas; multiple myelomas; chronic lymphocytic leukemias; and acute or chronic granulocytic lymphomas.

The novel compounds in accordance with the invention are particularly useful in the treatment of non-Hodgkin's lymphoma, multiple myeloma, melanoma, and ovarian, urothelial, oesophageal, lung, prostate, colon, gastric and breast cancers.

The compounds can be utilized to prevent or delay the appearance or reappearance, or to treat these pathological conditions. The compounds may be used as antiangiogenesis inhibitors for both anticancer activities or for abnormal wound healing or other hyperproliferative diseases dependent on blood vessel formation.

In addition, the compound of formula I is useful in treating and/or preventing polycystic kidney diseases (PKD) and rheumatoid arthritis. The compounds of this invention may also be useful for the treatment of Alzheimer's or Parkinson's disease or multiple sclerosis.

The crystal forms of the instant invention can be administered to a mammal in need of treatment therewith at dosage levels in the range of from about 0.5 to about 1000 mg/kg per day, preferably from about 5 to about 500 mg/kg per day. Other dose ranges include from about 5 to about 2000 mg/kg per week or twice a week, preferably from about 20 to about 1500 mg/kg per week or twice a week. The crystal forms of the instant invention may also be administered in a dosage range from about 5 to about 2500 mg/kg every three weeks, preferably from about 40 to about 2000 mg/kg every three weeks. However, some variability in the general dosage range may be required depending upon the age and weight of the subject being treated, the intended route of administration, and the like. The determination of dosage ranges and optimal dosages for a particular patient is well within the ability of one of ordinary skill in the art having benefit of the instant disclosure.

According to the methods of the invention, the crystal forms of the instant invention are administered to a mammal in need of treatment therewith, preferably in the form of a pharmaceutical composition comprising a pharmaceutically acceptable carrier, or excipient. Accordingly, such crystal forms can be administered to a mammal, for example, in oral, rectal, transdermal, parenteral, (e.g., intravenous, intramuscular, or subcutaneous), intracisternal, intravaginal, intraperitoneal, intravesical, local (e.g., powder, ointment, or drop), buccal, or nasal dosage form. Preferably, the crystal forms of the instant invention are administered to a mammal orally.

Compositions suitable for parenteral injection may comprise pharmaceutically acceptable sterile aqueous or nonaqueous solutions, dispersions, suspensions, emulsions, or mixtures thereof, and sterile powders for reconstitution into sterile injectable solutions or dispersions. Examples of suitable aqueous and nonaqueous carriers, diluents, solvents, or vehicles include water, ethanol, polyols (e.g., propylene glycol, polyethylene glycol, glycerol, and the like), suitable mixtures thereof, vegetable oils (e.g., olive oil), and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Prevention of microorganism contamination of these compositions can be effected with various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be affected by the use of agents capable of delaying absorption, for example, aluminum monostearate, and gelatin.

Solid dosage forms for oral administration include capsules, tablets, powders, and granules. In such dosage forms, the crystal forms of the instant invention are preferably admixed with at least one inert customary pharmaceutical excipient (or carrier) such as sodium citrate, or dicalcium phosphate, or (a) fillers or extenders; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, cetyl alcohol and glycerol monostearate; (g) adsorbents, as for example, kaolin and bentonite; and/or (h) lubricants, as for example, talc, calcium stearate, magnesium stearate, solid polyethylene glycols, sodium lauryl sulfate, or mixtures thereof. In the case of capsules and tablets, the dosages forms may also comprise buffering agents.

Solid compositions of a similar type may also be employed as fillers in soft or hard filled gelatin capsules using such excipients as lactose or milk sugar, as well as high molecular weight polyethylene glycols, and the like.

Solid dosage forms such as tablets, dragees, capsules, and granules can be prepared with coating and shells such as enteric coatings and others well known in the art. They may also contain certain opacifying agents, and can be of such composition that they release the active compound or compounds in a delayed manner. Examples of embedding compositions that can also be employed are polymeric substances and waxes. The crystal forms of the instant invention can also be incorporated in micro-encapsulated form, if appropriate, with one or more of the above-mentioned excipients.

Liquid dosage forms for oral administration include, for example, pharmaceutically acceptable emulsions, solutions, suspensions, syrups, and elixirs. In addition to the crystal forms of the instant invention, the liquid dosage form may contain inert diluents such as those commonly used in the art, e.g., water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oil, in particular, cottonseed oil, groundnut oil, corn germ oil, castor oil, and sesame seed oil, glycerol, tetrahydrofurfuryl alcohol, polyethylene glycols, and fatty acid esters of sorbitan, or mixtures of these substances, and the like.

Besides such inert diluents, the compositions may also comprise adjuvants, such as wetting agents, emulsifying and suspending agents, sweetening, flavoring, and perfuming agents.

Suspensions of the crystal forms of the instant invention may further comprise suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal or vaginal administration preferably comprise suppositories, which can be prepared by admixing the crystal forms of the instant invention with suitable non-irritating excipients or carriers such as cocoa butter, polyethylene glycol, or a suppository wax, which are solid at room temperature, but liquid at body temperature and, therefore, melt in the rectum or vaginal cavity thereby releasing such crystal forms.

Dosage forms for topical administration may comprise ointments, powders, sprays, and inhalants. The crystal forms of the instant invention are admixed under sterile conditions with a pharmaceutically acceptable carrier, and any preservatives, buffers, or propellants that may also be required. Opthalmic formulations, eye ointments, powders, and solutions are also intended to be included within the scope of the present invention.

### Experimental

Solid State Nuclear Magnetic Resonance (SSNMR)

All solid-state C-13 NMR measurements were made with a Bruker DSX-400, 400 MHz NMR spectrometer. High resolution spectra were obtained using high-power proton decoupling and the TPPM pulse sequence and ramp amplitude cross-polarization (RAMP-CP) with magic-angle spinning (MAS) at approximately 12 kHz (A. E. Bennett et al, J. Chem. Phys., 1995, 103, 6951), (G. Metz, X. Wu and S. O. Smith, J. Magn. Reson. A,. 1994, 110, 219-227). Approximately 70 mg of sample, 110 packed into a canister-design zirconia rotor was used for each experiment. Chemical shifts (δ) were referenced to external adamantane with the high frequency resonance being set to 38.56 ppm (W. L. Earl and D. L. VanderHart, J. Magn. Reson., 1982, 48, 35-54).

X-Ray Powder Diffraction (XRPD)

X-ray powder diffraction data for the crystalline forms of Compound (I) were obtained using a Bruker C2 GADDS system. The sample-detector distance was 15 cm. The radiation was CuKθ (40 kV, 50 mA). Data were collected at room temperature from 3 to 35 degrees 2θ with a sample exposure time of at least 2000 seconds. Powder samples were packed in glass capillaries (1 mm in diameter), and the capillary was rotated during data collection. The resulting two-dimensional diffraction arcs were integrated to create a traditional 1-dimensional PXRD pattern with a step size of 0.02 degrees 2θ in the range of 3 to 35 degrees 2θ.

Differential Scanning Calorimetry (DSC)

The DSC instrument used to test the crystalline forms was a TA Instruments® model Q1000. The DSC cell/sample chamber was purged with 100 ml/min of ultra-high purity nitrogen gas. The instrument was calibrated with high purity indium. The accuracy of the measured sample temperature with this method is within about +/−1° C., and the heat of fusion can be measured within a relative error of about +/−5%. The sample was placed into an open aluminum DSC pan and measured against an empty reference pan. About 2-6 mg of sample powder was placed into the bottom of the pan and lightly tapped down to make contact with the pan. The weight of the sample was measured accurately and recorded to a hundredth of a milligram. The instrument was programmed to heat at 10° C. per minute in the temperature range between 25 and 300° C. The heat flow, which was normalized by a sample weight, was plotted versus the measured sample temperature. The data were reported in units of watts/gram (“W/g”). The plot was made with the endothermic peaks pointing down. The endothermic melt peak was evaluated for extrapolated onset temperature, peak temperature, and heat of fusion in this analysis.

Thermogravimetric Analysis (TGA)

The TGA instrument used to test the crystalline forms was a TAInstruments® model Q500. The instrument was calibrated with potassium oxalate. The accuracy of the measured sample temperature with this method is within about +/−1° C. Samples of 15 to 20 milligrams were analyzed at a heating rate of 10° C. per minute in the temperature range between 25° C. and about 300° C.

Raman Spectroscopy

The Raman spectrum was acquired at a resolution of 4 cm⁻¹ with 128 co-added scans, using a Nicolet 950 FT-Raman spectrophotometer. The wavelength of the laser excitation was 1064 nm. A CaF₂ beam splitter and a Germanium, liquid nitrogen cooled detector were used.

Infrared Spectroscopy

The mid-IR spectra were collected using a Nicolet 560 FT-IR spectrophotometer by the KBr pellet, attenuated total reflectance and diffuse reflectance sampling techniques. These spectra are overlaid in FIG. 9. Upon comparison of the spectra to each other, the qualities of the spectra differ slightly depending upon the mode of sample preparation. In the IR spectra acquired via the attenuated total reflectance and diffuse reflectance techniques, well-resolved absorption bands are noted for Form N-2.

### EXAMPLES

The compound 3′-tert-Butyl-3′-N-tert-butyloxycarbonyl-4-deacetyl-3′-dephenyl-3′-N-debenzoyl-4-O-methoxycarbonyl-paclitaxel may be prepared according to the methodologies disclosed in the aforementioned U.S. Pat. No. 6,750,246. The following non-limiting examples serve to further illustrate the practice of the invention.

### Example 1

### Preparation of 3′-tert-Butyl-3′-N-tert-butyloxycarbonyl-4-deacetyl-3′-dephenyl-3′-N-debenzoyl-4-O-methoxycarbonyl-paclitaxel Form THF-1

A 2 L 3-necked flask equipped with a magnetic stirrer, argon inlet adapter, temperature probe and an addition funnel was flushed with argon. The 3′-tert-Butyl-3′-N-tert-butyloxycarbonyl-4-deacetyl-3′-dephenyl-3′-N-debenzoyl-4-O-methoxycarbonyl-paclitaxel (50 g) was charged into the flask followed by THF (250 ml). The mixture was heated to about 60° C. (internal temperature). Heptane (750 ml) was added slowly into the reactor while maintaining the internal temperature between about 55 to about 60° C. The mixture was then seeded with crystals of 3′-tert-Butyl-3′-N-tert-butyloxycarbonyl-4-deacetyl-3′-dephenyl-3′-N-debenzoyl-4-O-methoxycarbonyl-paclitaxel and cooled to about 50° C. The mixture was stirred at about 50° C. for about 4 hours, cooled to room temperature over 1.5 hours and stirred at room temperature for about 1.5 h. Filtration followed by drying at about 67° C. in an oven under vacuum and a flow of nitrogen for about 16 hours gave 36.5 g of the product as Form THF-1 (white solid, AP≧96.7%).

### Example 2

### Preparation of 3′-tert-Butyl-3′-N-tert-butyloxycarbonyl-4-deacetyl-3′-dephenyl-3′-N-debenzoyl-4-O-methoxycarbonyl-paclitaxel Form N-2

To a 50 ml, 3-necked flask equipped with a magnetic stirrer, argon inlet adapter and temperature probe was charged 3′-tert-Butyl-3′-N-tert-butyloxycarbonyl-4-deacetyl-3′-dephenyl-3′-N-debenzoyl-4-O-methoxycarbonyl-paclitaxel Form THF-1 as obtained from Example 1. Ethyl acetate (6.05 ml) was added to the flask and heated to about 60° C. (internal temperature). Heptane (18.15 ml) was added dropwise into the flask while maintaining the temperature between about 55 to about 60° C. The mixture was seeded with crystals of 3′-tert-Butyl-3′-N-tert-butyloxycarbonyl-4-deacetyl-3′-dephenyl-3′-N-debenzoyl-4-O-methoxycarbonyl-paclitaxel Form N-2 and stirred at about 60° C. for about 3.5 hours. The mixture was cooled to room temperature over 1.5 hour period and stirred at room temperature for about 1.5 hours. The solids were filtered and the cake was washed with 2 bed volumes of 1:3 ethyl acetate/heptane. Drying at 68-70° C. under vacuum and a flow of nitrogen gave 0.97 g (77% recovery) of Form N-2 as white solid (AP≧98.5%).

### Example 3

### Preparation of 3′-tert-Butyl-3′-N-tert-butyloxycarbonyl-4-deacetyl-3′-dephenyl-3′-N-debenzoyl-4-O-methoxycarbonyl-paclitaxel Capsule Formulations A

1. Add weighed amount of polyethylene glycol (PEG) 400 to a batching vessel pre-equilibrated to approximately 65° C. As an example, for a 5 kg batch size, add 1400 mg of PEG 400.

2. Add weighed amount high molecular weight polyethylene glycol (PEG) 1450 in powder, granular or pre-melted molten form to the batching vessel in step 1. As an example, for a 5 kg batch size, add 2800 mg of PEG 1450.

3. Add weighed amount of Tween 80 to the batching vessel from step 2 containing the PEG 400 and PEG 1450. As an example, for a 5 kg batch size, add 600 mg Tween 80.

4. Begin stirring to completely melt and mix the PEG/surfactant mixture from step 3 at approximately 65° C. to obtain a clear, homogeneous solution.

5. Optionally, add weighed amount of citric acid to the stirring PEG/surfactant mixture from step 4 and continue stirring at 65° C. until the acid dissolves. As an example, for a 5 kg batch size, add 5 mg citric acid.

6. Slowly add the weighed amount of 3′-tert-Butyl-3′-N-tert-butyloxycarbonyl-4-deacetyl-3′-dephenyl-3′-N-debenzoyl-4-O-methoxycarbonyl-paclitaxel Form N-2 to the stirring solution of PEG/surfactant/acid (if used) from step 5 with continuous stirring at 65° C. As an example, for a 5 kg batch size, add 200 g of 3′-tert-Butyl-3′-N-tert-butyloxycarbonyl-4-deacetyl-3′-dephenyl-3′-N-debenzoyl-4-O-methoxycarbonyl-paclitaxel Form N-2.

7. Continue stirring the PEG/surfactant/acid (if used)/3′-tert-Butyl-3′-N-tert-butyloxycarbonyl-4-deacetyl-3′-dephenyl-3′-N-debenzoyl-4-β-methoxycarbonyl-paclitaxel mixture from step 6 at approximately 65° C. to give a clear, homogeneous solution.

8. Fill an appropriate amount of the solution from step 7 into capsule shells to provide capsules of various dosage strengths.*
*For formulation solutions with a 4% w/w drug (i.e., 3′-tert-Butyl-3′-N-tert-butyloxycarbonyl-4-deacetyl-3′-dephenyl-3′-N-debenzoyl-4-O-methoxycarbonyl-paclitaxel) load, 5-mg strength and 25-mg strength capsules are prepared by filling 125 mg and 625 mg of the formulation solutions into Size #1 (or #2) and Size #0 two-piece hard gelatin capsule shells, respectively.

9. Allow the contents of the capsules from step 8 to solidify.

10. Place the caps on the filled capsule bodies from step 9.

### Example 4

### Preparation of 3′-tert-Butyl-3′-N-tert-butyloxycarbonyl-4-deacetyl-3′-dephenyl-3′-N-debenzoyl-4-O-methoxycarbonyl-paclitaxel Capsule Formulations B

1. Add weighed amount of polyethylene glycol (PEG) 3350 to a batching vessel pre-equilibrated to approximately 70° C. As an example, for a 5 kg batch size, add 4195 mg of PEG 3350.

2. Add weighed amount of Tween 80 to the batching vessel from step 1 containing the PEG 3350. As an example, for a 5 kg batch size, add 600 mg of Tween 80.

3. Begin stirring to completely melt and mix the PEG 3350/Tween 80 mixture from step 2 at approximately 70° C. to obtain a clear, homogeneous solution.

4. Add weighed amount of citric acid to the stirring PEG 3350/Tween 80 mixture from step 3 and continue stirring at 70° C. As an example, for a 5 kg batch size, add 5 mg of citric acid.

5. Continue stirring at approximately 70° C. to completely mix and dissolve the citric acid.

6. Slowly add the weighed amount of 3′-tert-Butyl-3′-N-tert-butyloxycarbonyl-4-deacetyl-3′-dephenyl-3′-N-debenzoyl-4-O-methoxycarbonyl-paclitaxel Form N-2 to the stirring PEG 3350/Tween 80/Citric acid solution from step 5 with continuous stirring at 70° C. As an example, for a 5 kg batch size, add 200 g of 3′-tert-Butyl-3′-N-tert-butyloxycarbonyl-4-deacetyl-3′-dephenyl-3′-N-debenzoyl-4-O-methoxycarbonyl-paclitaxel Form N-2.

7. Continue stirring the PEG 3350/Tween 80/Citric acid/3′-tert-Butyl-3′-N-tert-butyloxycarbonyl-4-deacetyl-3′-dephenyl-3′-N-debenzoyl-4-O-methoxycarbonyl-paclitaxel mixture from step 6 at approximately 70° C. to give a clear, homogeneous solution.

8. Fill an appropriate amount of the solution from step 7 into capsule shells to provide capsules of various dosage strengths*.
*For formulation solutions with a 4% w/w drug (e.g., 3′-tert-Butyl-3′-N-tert-butyloxycarbonyl-4-deacetyl-3′-dephenyl-3′-N-debenzoyl-4-O-methoxycarbonyl-paclitaxel) load, 5-mg strength and 25-mg strength capsules are prepared by filling 125 mg and 625 mg of the formulation solutions into Size #2 and Size #0 two-piece hard gelatin capsule shells, respectively.

9. Allow the contents of the capsules from step 8 to solidify.

10. Place the caps on the filled capsule bodies from step 10.

We claim:

1. A crystalline form of 3′-tert-Butyl-3′-N-tert-butyloxycarbonyl-4-deacetyl-3′-dephenyl-3′-N-debenzoyl-4-O-methoxycarbonyl-paclitaxel represented by formula I:

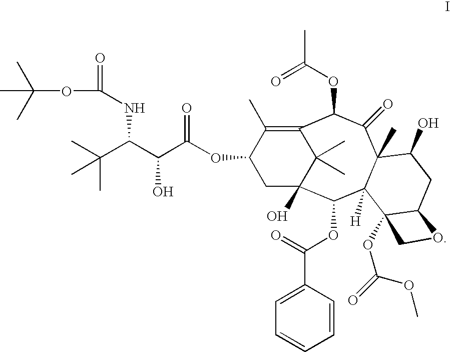

2. The crystalline form of claim 1 which is substantially pure.

3. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a powder X-ray diffraction pattern comprising peaks expressed in degrees two-theta at approximately 3.0±0.2, 6.2±0.2, 8.1±0.2, 9.2±0.2, 10.0±0.2, 13.5±0.2 and 16.4±0.2.

4. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a powder X-ray diffraction pattern substantially the same as shown in FIG. 1.

5. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a ¹³C solid state nuclear magnetic resonance comprising chemical shifts expressed in part per million at approximately 10.1, 15.1, 16.4, 19.7, 21.5, 23.5, 24.1, 25.2, 25.7, 26.8, 27.6, 29.1, 35.0, 35.3, 36.0, 36.3, 36.6, 43.4, 45.0, 46.5, 56.4, 57.3, 58.7, 61.0, 62.6, 80.7, 82.5, 83.3, 83.6, 129.1, 129.7, 130.6, 131.7, 133.4, 133.5, 133.9, 134.4, 143.2, 147.0, 154.8, 155.5, 156.9, 168.7, 169.0, 173.1, 173.6, 175.9, and 176.7.

6. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a ¹³C solid state nuclear magnetic resonance spectrum substantially the same as shown in FIG. 7.

7. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a Raman spectrum comprising frequencies expressed in cm⁻¹ at approximately 3069, 3027, 2975, 2961, 2938, 2864, 1750, 1709, 1602, 1585, 1453, 1165, 1060, 1000, 904, 854, and 617.

8. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a Raman spectrum substantially the same as shown in FIG. 8.

9. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits IR spectra substantially the same as shown in FIG. 9.

10. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a TGA thermogram substantially the same as shown in FIG. 5.

11. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form N-2) which exhibits a DSC thermogram substantially the same as shown in FIG. 3.

12. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form THF-1) which exhibits a powder X-ray diffraction pattern comprising peaks expressed in degrees two-theta at approximately 6.0±0.2, 10.7±0.2, 11.5±0.2, 12.0±0.2, 16.8±0.2, and 19.5±0.2.

13. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form THF-1) which exhibits a powder X-ray diffraction pattern substantially the same as shown in FIG. 2.

14. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form THF-1) which exhibits a TGA thermogram substantially the same as shown in FIG. 6.

15. A crystalline form of 3′-tert-Butyl-3′-N-tert-butyloxycarbonyl-4-deacetyl-3′-dephenyl-3′-N-debenzoyl-4-O-methoxycarbonyl-paclitaxel (Form THF-1) which exhibits a DSC thermogram substantially the same as shown in FIG. 4.

16. A process for preparing 3′-tert-Butyl-3′-N-tert-butyloxycarbonyl-4-deacetyl-3′-dephenyl-3′-N-debenzoyl-4-O-methoxycarbonyl-paclitaxel (Form THF-1), which process comprises the steps of:
(a) mixing 3′-tert-Butyl-3′-N-tert-butyloxycarbonyl-4-deacetyl-3′-dephenyl-3′-N-debenzoyl-4-O-methoxycarbonyl-paclitaxel in an aprotic solvent system with heating until dissolution is essentially complete to form a solution, and said aprotic solvent system comprises (i) THF; and (ii) at least one solvent selected from heptane, hexane, cyclohexane, and toluene; and
(b) cooling the resulting solution to a lower temperature to allow Form THF-1 to crystallize.

17. The process of claim 16, wherein said lower temperature is room temperature.

18. A process for preparing 3′-tert-Butyl-3′-N-tert-butyloxycarbonyl-4-deacetyl-3′-dephenyl-3′-N-debenzoyl-4-O-methoxycarbonyl-paclitaxel (Form N-2), which process comprises the steps of:
(a) mixing 3′-tert-Butyl-3′-N-tert-butyloxycarbonyl-4-deacetyl-3′-dephenyl-3′-N-debenzoyl-4-O-methoxycarbonyl-paclitaxel or Form THF-1 of 3′-tert-Butyl-3′-N-tert-butyloxycarbonyl-4-deacetyl-3′-dephenyl-3′-N-debenzoyl-4-O-methoxycarbonyl-paclitaxel in an aprotic solvent system with heating until dissolution is essentially complete to form a solution, and said aprotic solvent system comprises (i) at least one solvent selected from ethyl acetate, isopropyl acetate, and toluene; and (ii) at least one solvent selected from heptane, hexane, and cyclohexane; and
(b) cooling the resulting solution to a lower temperature to allow Form N-2 to crystallize.

19. The process of claim 18, wherein said aprotic solvent system comprises (i) ethyl acetate; and (ii) at least one solvent selected from heptane, hexane, and cyclohexane; and wherein said lower temperature is room temperature.

20. A process for preparing a pharmaceutical formulation containing 3′-tert-Butyl-3′-N-tert-butyloxycarbonyl-4-deacetyl-3′-dephenyl-3′-N-debenzoyl-4-O-methoxycarbonyl-paclitaxel and at least one pharmaceutically acceptable carrier or excipient, which process comprises mixing Form N-2 crystals of 3′-tert-Butyl-3′-N-tert-butyloxycarbonyl-4-deacetyl-3′-dephenyl-3′-N-debenzoyl-4-O-methoxycarbonyl-paclitaxel with at least one said pharmaceutically acceptable carrier or excipient.

21. A pharmaceutical composition comprising Form N-2 or Form THF-1 crystals of 3′-tert-Butyl-3′-N-tert-butyloxycarbonyl-4-deacetyl-3′-dephenyl-3′-N-debenzoyl-4-O-methoxycarbonyl-paclitaxel and at least one pharmaceutically acceptable carrier or excipient.

22. A method for inhibiting tumor growth which method comprises administering to a mammal in need of such treatment a Form N-2 crystal of 3′-tert-Butyl-3′-N-tert-butyloxycarbonyl-4-deacetyl-3′-dephenyl-3′-N-debenzoyl-4-O- methoxycarbonyl-paclitaxel, a Form THF-1 crystal of 3′-tert-Butyl-3′-N-tert-butyloxycarbonyl-4-deacetyl-3′-dephenyl-3′-N-debenzoyl-4-O-methoxycarbonyl-paclitaxel, or a pharmaceutical composition comprising such Form N-2 or Form THF-1 crystal.

23. The method of claim 22, which method comprises administering to a mammal in need of such treatment a Form N-2 crystal of 3′-tert-Butyl-3′-N-tert-butyloxycarbonyl-4-deacetyl-3′-dephenyl-3′-N-debenzoyl-4-O-methoxycarbonyl-paclitaxel, or a pharmaceutical composition comprising such Form N-2 crystal.

24. The method of claim 23, wherein said Form N-2 crystal is administered orally.

25. 3′-tert-Butyl-3′-N-tert-butyloxycarbonyl-4-deacetyl-3′-dephenyl-3′-N-debenzoyl-4-O-methoxycarbonyl-paclitaxel in a form consisting essentially of crystalline 3′-tert-Butyl-3′-N-tert-butyloxycarbonyl-4-deacetyl-3′-dephenyl-3′-N-debenzoyl-4-O-methoxycarbonyl-paclitaxel.

26. A pharmaceutical composition comprising 3′-tert-Butyl-3′-N-tert-butyloxycarbonyl-4-deacetyl-3′-dephenyl-3′-N-debenzoyl-4-O-methoxycarbonyl-paclitaxel in a form consisting essentially of crystalline 3′-tert-Butyl-3′-N-tert-butyloxycarbonyl-4-deacetyl-3′-dephenyl-3′-N-debenzoyl-4-O-methoxycarbonyl-paclitaxel.
